(19) United States
(12) Patent Application Publication
(10) Pub. No.: US 2010/0310653 A1
(43) Pub. Date: Dec. 9, 2010

(54) USE OF AN EXTRACT MADE OF LEAVES OF GINKGO BILOBA

(75) Inventors: Joachim Herrmann, St. Leon-Rot (DE); Robert Hörr, Karlsruhe (DE)

Correspondence Address: EDWARDS ANGELL PALMER & DODGE LLP, P.O. BOX 55874, BOSTON, MA, 02205, US

(73) Assignee: DR. WILLMAR SCHWABE GMBH & CO. KG, Karlsruhe (DE)

(21) Appl. No.: 12/809,236

(22) PCT Filed: Dec. 18, 2008
(86) PCT No.: PCT/EP2008/010799

§ 371 Date: Aug. 23, 2010

(30) Foreign Application Priority Data
Dec. 21, 2007 (EP) EP07024973.5

(51) Int. Cl. A61K 36/16; A61K 9/28; A61K 9/22; A61P 25/28

(52) U.S. Cl. 424/468; 424/752; 424/474

(57) ABSTRACT

The present invention relates to the use of an extract made of leaves of Ginkgo biloba for the production of a preparation, or an extract made of leaves of Ginkgo biloba as an agent for the treatment and prophylaxis of the dementia syndrome, the early stages and pre-stages thereof, wherein 180 to 300 mg of Ginkgo extract is administered once a day.

The present invention relates to the use of an extract made of leaves of Ginkgo biloba for the production of an agent for the treatment and prevention of the dementia syndrome, early stages and pre-stages thereof, wherein 180 to 300 mg of Ginkgo extract is administered once per day. Or in other terms, this invention relates to an extract made of leaves of Ginkgo biloba as an agent for the treatment and prevention of the dementia syndrome and the early stages and pre-stages thereof, wherein 180 to 300 mg of Ginkgo extract is administered once per day.

Since decades, extracts from the leaves of Ginkgo biloba are used as a medicament. They are currently used for the treatment of different kinds of dementia and symptoms thereof as well as cerebral and peripheral blood circulation disorders. Ingredients, the efficacy is associated with, are terpene lactones (ginkgolides A, B, C and bilobalide) as well as glycosides of flavones (quercetin, kaempferol and isorhamnetin).

Most Ginkgo extracts for pharmaceutical use are standardized by a content of 22.0 to 27.0% by weight of glycosides of flavones, 5.0 to 7.0% by weight of terpene lactones and 5 ppm ginkgolic acids at the most, with a ratio drogue to extract of 35 to 67 to 1. The special extract EGb761® contained in Tebonin® complies with this specification.

28 entries for the active agent Ginkgo and in total 54 different products are present in the Rote Liste (online edition 2007). 34 of those are monoproducts containing extract from leaves and the remaining 20 are homeopathic drugs, containing tinctures or dilutions, produced according to homeopathic guidelines. Film-coated tablets prevail the presentation forms of extracts made of leaves of Gingko, furthermore there is one dragee and nine liquids (drops). The film-coated tablets contain 40, 50, 60, 80 or 120 mg of extract made of leaves of Ginkgo biloba.

Commission E is an autonomous, scientific commission of the former deutsches Bundesgesundheitsamt (BGA), nowadays Bundesinstitut für Arzneimittel und Medizinprodukte (BfArM). Throughout the years 1980 to 1994 the task of Commission E was to gather, elaborate and evaluate material both, scientific material and empirically medical material, regarding desired and unwanted effects of herbal drogues. The created monographs are still valid and are foundation for new approvals and post-approvals of herbal drugs.

The corresponding Commission E monograph for a dry extract made of leaves of Ginkgo biloba, published in Bundesanzeiger Nr. 133 on 19.07.1994, recommends a total daily dose of 120-240 mg of dry extract, administered as two or three single doses, for the treatment of the dementia syndrome. All products comply with this recommendation, except Gingium 120 intens, a film-coated tablet of which with 120 mg of Ginkgo extract is to be administered 1-2 times per day.

For all other indications except the dementia syndrome, smaller doses are recommended.

An issue at the treatment of diseases by medicaments is the poor compliance, concerning the intake of the drugs. It is known that reliability of the intake decreases with the number of daily doses. Accordingly one object at the development of drugs is a low number of daily doses, with a single daily intake at the same time each day resulting in the optimum compliance. Reciprocally, several intakes a day produce risk of single intakes being forgotten, resulting in a lower efficacy of the drug. This is especially important for the treatment of the dementia syndrome, characterized by impairments of the memory and concentration, reduced ability of daily living skill and a thus significantly reduced quality of life.

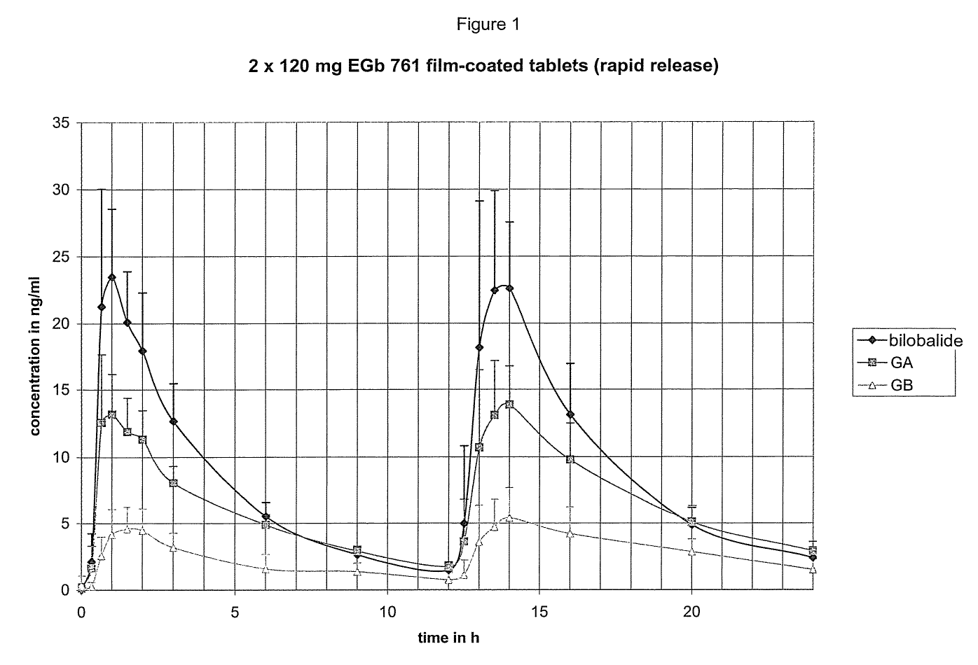

The aim of development of drugs with a single daily intake is opposed by the pharmacokinetic nature of many drugs, which after uptake into the blood system have a short time of presence or short half life time of elimination. This requires several intakes per day to maintain an effective concentration of the drug. This applies to the extract made of leaves of Ginkgo as well, for which corresponding trials (FIG. 1) show, that very little concentrations of the effectiveness determining lead substances bilobalide, ginkgolid A (GA) and ginkgolid B (GB) are found after 12 hours and require a new intake.

FIG. 1 shows the concentration of bilobalide, ginkgolid A (GA) und ginkgolid B (GB) in the blood plasma of humans against time, wherein one tablet containing 120 mg Ginkgo extract is administered at the beginning and after 12 hours. The presented values are means of 12 test persons. The administered amount is equal to the daily maximum dose, recommended by the monograph.

Thus the aim of this invention is the improvement of the treatment of the dementia syndrome with extracts made of Gingko.

Although it was not expected due to the short half life, it was found now, that important parameters (like “improvement of quality of life” and “for outsiders noticeable global improvement”), which are measured during the evaluation of effectiveness of antidementives and which are especially important as indicators for the clinical relevance of the effects of treatment, were more improved at a single daily intake of a 240 mg tablet than at the administration of one 120 mg tablet twice a day. As shown in the examples, effects were achieved with the administration of one film-coated tablet with 240 mg EGb 761® once per day, which could not be shown with other dosage forms at a partitioning into two doses of each 120 mg per day. It is thus proven, that the film-coated tablet with 240 mg EGb 761® using an oral administration once a day, has an exceeding, independent, therapeutic advantage compared to other dosage forms.

Subject of the invention is accordingly the use of an extract made of leaves of Ginkgo biloba for the production of an agent for the treatment and prevention of the dementia syndrome, the early stages and pre-stages thereof, wherein 180 to 300 mg, preferably 220 to 260 mg and most preferred 240 mg Ginkgo extract is administered once per day. The agent can be a drug or a food product, like functional/medical food, dietetic food or novel food.

In other terms, this invention concerns an extract made of leaves of Ginkgo biloba for the production of an agent for the treatment and prevention of the dementia syndrome, the early and preliminary stages thereof, wherein 180 to 300 mg, preferably 220 to 260 mg and most preferred 240 mg Ginkgo extract is administered once per day. The agent can be a drug or food product, like functional/medical food, dietetic food or novel food.

Furthermore subject of the invention is the use of an extract made of leaves of Ginkgo biloba as a food product like e. g. a dietary supplement, functional/medical food, dietetic food or novel food for the treatment and prevention of the dementia syndrome, the early stages and pre-stages thereof, wherein 180 to 300 mg, preferably 220 to 260 mg and most preferred 240 mg Ginkgo extract is administered once per day.

The dementia syndrome (also called dementia) is defined as an acquired impairment of the memory and one or several further cognitive functions to such an extent, that activities of everyday life and/or social relationships are severely impaired. Apart from impairments of the memory, the dementia syndrome causes symptoms such as disorders of concentration, depressive disorders, dizziness, buzzing in one's ears and headache. Especially primary neurodegeneration at Alzheimer disease and vascular reasons (disorders of cerebral blood flow) are considered as causes for the dementia syndrome. The most common forms of are accordingly Alzheimer dementia (also called primary degenerative dementia of Alzheimer type), vascular dementia (e.g. multi-infarct dementia) and mixed forms of both. With the help of sensitive tests, it is possible to recognize early stages and pre-stages of dementia, which do not fulfill the internationally accepted diagnostic criteria for dementia yet, but already show recognizable impairments of cognitive performance. Those are characterized as mild cognitive impairment (MCI) or cognitive impairment, which is not dementia yet, no dementia (CIND). Age related symptomatic complexes, which can represent pre-stages of dementia, comprise different combinations of two or several of the following symptoms: subjectively perceived impairments of the memory, subjective impairments of other cognitive components of performance (e.g. attention, concentration, ability of verbal expression, ability and velocity to solve problems, ability of spatial imagination, planning, conceptional thinking and carrying out of complex activities etc.), objectively impaired memory performance, objective impairments of other cognitive components of performance (e.g. attention, concentration, ability of verbal expression, ability and velocity to solve problems, ability of spatial imagination, planning, conceptional thinking and carrying out of complex activities etc.), depressive disorder, anxiety, depressive disorder, apathy, lack of motivation, indifference, irritability, excitement, sleeping disorders and disorders of day and night rhythm.

Preferred are Ginkgo extracts according to the specifications of DAB 2003 and the not valid yet, but already published European Pharmacopeia 6.1. Ginkgo extracts according to DAB 2003 are produced using acetone 60% (m/m) and a mulit-stage extraction method, with the ration drug to extract (DEV) being between 35 and 67 to 1. Those Ginkgo extracts are characterized by a content of flavonoids of at least 22.0% and at most 27.0%, of terpene lactones of at least 5.0% and at most 7.0% (of which 2.8 to 3.4% ginkgolids A, B and C and 2.6 to 3.2% bilobalide) and of ginkgolic acid of 5 ppm at the most. Ginkgo extracts according to European Pharmacopeia 6.1 are produced with organic solvents and their mixtures with water, with the ratio of drug to extract (DEV) not being specified and contents of flavonoids of at least 22.0% and at most 27.0%, of bilobalide of 2.6 to 3.2%, of ginkgolids A, B and C of 2.8 to 3.4% and of ginkgolic acids of 5 ppm at the most. Especially preferred is the Ginkgo extract with the name EGb® 761, which is produced according to DAB 2003 using acetone 60% (m/m) and a multi-stage extraction method, with the ration drug to extract (DEV) being between 35 and 67 to 1. Those ginkgo extracts are characterized by a content of flavonoids of at least 22.0% and at most 27.0%, of terpene lactones of at least 5.0% and at most 7.0% (of which 2.8 to 3.4% ginkgolids A, B and C and 2.6 to 3.2% bilobalide) and of ginkgolic acids of 5 ppm at the most. Thus and due to the mentioned properties. EGb® 761 complies with European Pharmacopeia 6.1 as well. Though it is not specifically stated in DAB 2003 and European Pharmacopeia 6.1, all preceding percentage declarations are percent by weight. As well especially preferred are ginkgo extracts according to WO2006/117169, which furthermore have a reduced content of biflavones and/or 4′-O-methyl pyridoxine (<20 ppm, preferred <10 ppm, especially preferred <2 ppm). The invention, however is not restricted on these extracts.

The especially preferred ginkgo extract EGb® 761 can be produced for example according to the procedure generally described in EP431535B1 or according to the further optimized procedure described in WO2006/117170. For this
(a) dried and chopped leaves from ginkgo biloba are extracted at a temperature of 40 to 60° C. using aqueous acetone (approximately 60% by weight acetone),
(b) the acetone is removed to a maximal content of 5% by weight,
(c) the remaining concentrated aqueous solution is diluted with water to a content of solids of 10% by weight at the most, this solution is cooled to a temperature of 12° C. and the resulting precipitate is removed,
(d) ammonium sulfate (approximately 30% by weight) is added to the remaining aqueous solution and the resulting solution is extracted with methyl ethyl ketons or a mixture of methyl ethyl ketons and acetone (6/4),
(e) the resulted organic phase is concentrated and the obtained concentrate is diluted with water and ethanol to obtain a solution, which contains approximately 50% by weight water, approximately 50% by weight ethanol and approximately 10% by weight solids,
(f) an aqueous solution of lead hydroxyl acetate is added to the thus obtained solution and the obtained precipitate is removed,
(g) the remaining aqueous ethanol solution is extracted with heptane, to further remove the alkyl phenol compounds,
(h) the remaining aqueous-ethanolic solution is concentrated at reduced pressure and approximately 20% by weight ammonium sulfate is added,
(i) the obtained solution is extracted with a mixture of methy ethyl ketons and ethanol (6/4),
(k) the obtained organic phase is dried with at most 20% by weight ammonium sulfate and is concentrated, and ethanol is added to such an extent, that the ethanol content is at least 80% by weight. The temperature of the solution is kept below 12° C. for 2 to 12 hours and filtered,
(l) the obtained filtrate is concentrated at reduced pressure and is dried, to obtain a dry extract with a water content of less than 5%.

For the production of the especially preferred ginkgo extracts according to WO2006/117169 with a reduced content of biflavones and/or 4′-O-methyl pyridoxine e.g. the solution of above's step (k) is when necessary diluted with aqueous ethanol, filtered on adsorber resin and/or ion exchanger, where the desired substances are retained and dried with reduced pressure to a dry extract with a water content of less than 5%. The removal of biflavones and/or 4′-O-methyl pyridoxine doesn't result in a significant reduction in the content of the effective components.

The Ginkgo extracts useable according to this invention can be administered preferably orally in form of powders, granulates, effervescent preparations, tablets, dragees, capsules, or liquids. For the production of tablets, the extract is mixed with appropriate, pharmaceutically acceptable excipients such as lactose, cellulose, silicium dioxide, croscarmellose and magnesium stearate, pressed to tablets, which, if necessary, are coated with an appropriate film, e.g. made of hydroxypropylmethylcellulose, polyethylenglykol, colorants (e.g. titan dioxide) and talcum. The Ginkgo extracts useable according to this invention can be filled into capsules as well, if necessary using excipients like fillers; flow regulators, etc.

In the case of tablets, film-coated tablets are preferred. Furthermore those preparations are preferred, which release the extract quickly and are named in the European Pharmacopeia 6.0 “Conventional-release dosage forms”.

Especially preferred embodiments of this invention are:

Use of an extract made of leaves of Ginkgo biloba for the production of an agent or extract made of Ginkgo biloba as agent for the treatment and prevention of the dementia syndrome, early stages and pre-stages thereof, wherein 180 to 300 mg of ginkgo extract is administered, preferably orally once per day, wherein the extract contains 22.0 to 27.0% by weight of flavonoids, 2.6 to 3.2% by weight of bilobalide and 2.8 to 3.4% by weight of ginkgolids A, B and C (as sum). Preferably, the extract contains 5 ppm ginkgolic acid of at the most. Furthermore the extract preferably contains less than 20 ppm, especially preferably less than 10 ppm and most preferred less than 2 ppm 4′-O-methyl pyridoxine.

The extract with the composition stated above is administered orally once per day, preferably at a dose of 180 to 300 mg, more preferably at a dose of 220 to 260 mg and most preferably at a dose of 240 mg.

A particularly preferred embodiment of the present invention is the use of an extract made of leaves of Ginkgo biloba for the production of an agent or the extract made from Ginkgo biloba as agent for the treatment and prevention of the dementia syndrome, early stages and pre-stages thereof, wherein 240 mg of Ginkgo extract is administered orally once per day and the Ginkgo extract is the known extractEGb 761®. The dementia syndrome is preferably light or moderate dementia or a light to moderate dementia.

### EXAMPLES

The extract called EGb 761® below is a special extract of Dr. Willmar Schwabe GmbH & Co. KG made of leaves of ginkgo biloba in accordance with the guidelines established by the German Pharmacopoeia (DAB). It has a 22.0 to 27.0 wt.-% content of flavonoids, a 5.0 to 7 wt.-% content of terpene lactones (of those 2.8 to 3.4 wt.-% of ginkgolides A, B, and C and 2.6 to 3.2 wt.-% of bilobalide) and a maximum of 5 ppm of ginkgolic acids. These contents were determined according to the DAB, the flavonoids being confirmed as quercetin, kaempherol and isorhamnetin after acid hydrolysis and being calculated as glycosides of flavonoids.

### Comparative Example 1

### Improvement of the Quality of Life

The quality of life of patients was investigated in a randomised, placebo-controlled double blind study with EGb 761® on patients with dementia syndrome (mild to moderate dementia) [Schneider et al. 2005]. In this study, EGb 761® was used in the form of film-coated tablets of 60 mg or 120 mg, respectively, administering two tablets each per day to arrive at a daily dosage of 120 mg or 240 mg, respectively. Under this regime, none of the tested doses of EGb 761® was able to show an effect on the quality of life significant in comparison to a placebo when applying the Progressive Deterioration Scale (PDS) [De Jong et al. 1989], a validated scale for assessing the quality of life especially developed for patients with dementia (p=0.7).

### Example 1 of the Invention

### Improvement of the Quality of Life

By treating patients with dementia syndrome (mild to moderate dementia) with the fast-release film-coated tablet containing 240 mg of EGb 761® (once a day), an improvement in the quality of life of the patients treated with EGb 761® could be confirmed in a randomised, placebo-controlled double-blind study for the first time. When treated with the film-coated tablet at 240 mg of EGb 761° at a dosage of one tablet per day, an improvement of the quality of life was observed during a randomised therapy over 24 weeks which became manifest by an average increase in the total value of the validated Scale of the Quality of Life especially developed for dementia patients DEMQOL-Proxy [Smith et al. 2005] by 3.38 (s=8.45) points, while a noticeably smaller improvement by 1.36 (s=6.62) points was observed for the placebo group. The difference between the two treatment groups was statistically significant at p=0.004. When observing the progress over the 24 weeks of treatment, one will note that the mean treatment effect increases continuously with EGb 761® (1×240 mg) while there is only a slight temporary improvement in the placebo group which is often observed by the greater attention in connection with a clinical test, but which is then lost even during the observation period due to the natural progression of the disease [Walach et al. 2005] Looking at the results at patient level, one will recognise improvements of the quality of life after 24 weeks of treatment in 113 (55.9%) of the patients treated with EGb 761® (1×240 mg) but only in 90 of the patients given a placebo (44.6%) (p<0.05).

For the first time, it was possible to show a significant improvement of patients having dementia syndrome (mild to moderate dementia) by treating them with the 240 mg film-coated tablet of EGb 761® administered once a day in a randomised controlled clinical test. Moreover, there was a marked improvement of the quality of life vis-á-vis 2×60 mg or 2×120 mg of EGb 761® (comparative example 1).

### Comparative Example 2

### Global Improvement Evident to Outsiders

The global change in the condition of patients was also assessed by independent, unbiased outsiders on the basis of a validated Clinical Global Impression of Change scale (ADCS-CGIC) developed by the Alzheimer's Disease Cooperative Study in a randomised, placebo-controlled double-blind study with EGb 761® on patients with dementia syndrome (mild to moderate dementia) [Schneider et al. 2005]. In this study, no statistically significant advantage of a treatment with EGb 761® in the dosages of 2×60 mg and 2×120 mg per day was evident (p=0.2).

### Example of the Invention 2

### Global Improvement Evident to Outsiders

For the first time, a global improvement in the condition was noted in patients with dementia syndrome (mild to moderate dementia) who were treated with the 240 mg fast-release film-coated tablet (once a day) which was so pronounced that it was clearly evident to independent evaluators who were neither involved in the treatment of the patients nor aware of the findings of the medical examination, but talked to the patients and their families. This assessment by independent outsiders was also carried out on the basis of the validated Clinical Global Impression of Change Scale (ADCS-CGIC) [Schneider et al. 1997] developed by the Alzheimer's Disease Cooperative Study. This scale comprises 7 categories from “pronounced improvement” (value in the scale 1) via “no change” (value in the scale 4) to “pronounced deterioration” (value in the scale 7). A global improvement of the overall condition was noted in 109 patients (54.0%) treated with EGb 761® (1×240 mg), but only in 52 patients (25.7%) treated with a placebo (p>0.0001). Considering that the condition of dementia patients will inevitably deteriorate with time and that the non-occurrence of such a deterioration may already be rated as a success of the therapy, 186 (92.1%) were found among the patients treated with EGb 761® (1×240 mg) and 135 patients (66.8%) in the placebo group who did not deteriorate during the 24 week period of treatment (p<0.0001).

As a result of the treatment with the 240 mg film-coated tablet of EGb 761® once a day, a significantly improved assessment of the change in the global condition by independent outsiders not involved in the treatment of the patients and not aware of the findings of the medical examination in a randomised controlled clinical test was possible for the first time. In addition, there was a clear improvement in the change in the global condition vis-á-vis the administration of 2×60 mg or 2×120 mg of EGb 761® (Comparative example 2).

### Example 3 of the Invention

### 240 mg Tablet for Oral Administration Once a Day (“Conventional-Release Dosage Form” According to the European Pharmacopeia 6.0)

| Component | Amount in mg per tablet |
|---|---|
| Extract of leaves of Ginkgo EGb 761 ® | 240.0 |
| Lactose monohydrate | 160.0 |
| Microcrystalline cellulose | 290.0 |
| Corn starch | 50.0 |
| Highly disperse silica | 10.0 |
| Croscarmellose sodium | 40.0 |
| Magnesium stearate | 10.0 |
| Hypromellose | 20.0 |
| Macrogol | 5.0 |
| Talcum | 2.5 |
| Titania | 1.0 |

The extract made of leaves of Ginkgo is mixed with the lactose (filler), the microcrystalline cellulose (filler/binder), the corn starch (binder), the crosscarmellose (disintegrant) and the highly disperse silica (flow promoter) in one step in a suitable mixer. The magnesium stearate (lubricant) is added and mixing repeated briefly. This mixture is pressed in a rotary tablet press to form oval, convex tablets having a mean weight of 800 mg, a length of 17 mm and a width of 8 mm.

Hypromellose (coating agent) and macrogol (softener) are dissolved in purified water with stirring and talcum (anti-adhesive) and titania (colouring pigment) are added and dispersed with stirring. For swelling and complete dissolution of the polymer, this mixture is allowed to stand for 12 hours and then sprayed onto the tablets in a drum coater. The final product are film-coated tablets of a white colour for oral administration which contain 240 mg of extract of leaves of Ginkgo each.

### REFERENCES

DeJong R, Osterlund O W, Roy G W. Measurement of Quality-of-Life changes in patients with Alzheimer's disease. Clinical Therapeutics 1989; 11:545-554.
Schneider L S, Olin J T, Doody R S, Clark C M, Morris J C, Reisberg B, Schmitt F A, Grundman M, Thomas R G, Ferris S H, and the Alzheimer's Disease Cooperative Study. Validity and Reliability of the Alzheimer's Disease Cooperative Study—Clinical Global Impression of Change. Alzheimer Disease and Associated Disorders 1997; 11(suppl 2):S22-S32.
Schneider L S, DeKosky S T, Farlow M R, Tariot P N, Hoerr R, Kieser M. A randomized, double-blind, placebo-controlled trial of two doses of Ginkgo biloba extract in dementia of the Alzheimer's type. Current Alzheimer Research 2005; 2:541-551.
Smith S C, Lamping D L, Banerjee S. Harwood R. Foley B. Smith P. Cook J C, Murray J, Prince M, Levin E, Mann A, Knapp M. Measurement of health-related quality of life for people with dementia: development of anew instrument (DEW:20W and an evaluation of current methodology. Health Technology Assessment 2005; 9(10).
Walach H. Sadaghiani C. Dehm C. Bierman D. The therapeutic effect of clinical trials: understanding placebo response rates in clinical trials—A secondary analysis. BMC Medical Research Methodology 2005; 5:26.

1. Use of an extract made of leaves of Ginkgo biloba for the production of an agent or an extract made of leaves of Ginkgo biloba as an agent for treatment and prevention of the dementia syndrome, the early stages and pre-stages thereof, wherein 180 to 300 mg of Ginkgo extract is administered once per day.

2. Use or extract according to claim 1, wherein the dementia syndrome is selected from light dementia, moderate dementia, Alzheimer dementia, vascular dementia and their mixed forms.

3. Use or extract according to claim 1, wherein the early stages and pre-stages of dementia are light cognitive impairments (MCI), cognitive impairments, which are not dementia yet (CIND), subjectively perceived impairments of the memory, subjective impairments of other cognitive components of performance, objectively impaired memory performance, objective impairments of other cognitive components of performance, depressive disorder, anxiety, frightened mood, apathy, lack of motivation, indifference, irritability, excitement, sleeping disorders and disorders of day and night rhythm.

4. Use or extract according to claim 3, wherein the other subjectively and/or objectively impaired cognitive components of performance are selected from attention, concentration, ability of verbal expression, ability and velocity to solve problems, ability of spatial imagination, planning, conceptional thinking and carrying out of complex activities.

5. Use or extract according to claim 1, wherein the extract contains glycosides of flavones and terpene lactones.

6. Use or extract according to claim 5, wherein the extract contains at least 22.0% by weight and at most 27% by weight of flavonoids and at least 5.0% by weight and at most 7.0% by weight of terpene lactones.

7. Use or extract according to claim 5, wherein the extract contains 22.0% to 27.0% by weight of flavonoids, 2.6% to 3.2% by weight of bilobalide and 2.8% to 3.4% by weight of ginkgolids A, B and C (as sum).

8. Use or extract according to claim 1, wherein the extract contains 5 ppm ginkgolic acids at the most.

9. Use or extract according to claim 1, wherein the extract contains less than 20 ppm 4′-O-methyl pyridoxine.

10. Use or extract according to claim 9, wherein the extract contains less than 10 ppm 4′-O-methyl pyridoxine.

11. Use or extract according to claim 9, wherein the extract contains less than 2 ppm 4′-O-methyl pyridoxine.

12. Use or extract according to claim 1, wherein the extract is administrated in form of powders, granulates, effervescent preparations, tablets, dragees, capsules or liquids.

13. Use or extract according to claim 12, wherein the administration is oral.

14. Use or extract according to claim 12, wherein the tablet is a film-coated tablet.

15. Use or extract according to claim 12, wherein the tablet is a rapid release tablet.

16. Use or extract according to claim 1, wherein 220 to 260 mg of Ginkgo extract is administrated once per day.

17. Use or extract according to claim 1, wherein 240 mg of Ginkgo extract are administrated once per day.

18. Use or extract according to claim 1, wherein the extract EGb 761® is administrated in form of an agent orally once per day to treat light, moderate or light to moderate dementia.

19. Use or extract according to claim 1, wherein the agent is a drug.

20. Use or extract according to claim 1, wherein the agent is a food product.

21. Use or extract according to claim 20, wherein the food product is a dietary supplement, a functional/medical food, a dietetic food or a novel food.
